(19) United States
(12) Patent Application Publication
(10) Pub. No.: US 2011/0288001 A1
(43) Pub. Date: Nov. 24, 2011

(54) BIOLOGICALLY ACTIVE PROTEINS ACTIVATABLE BY PEPTIDASE

(76) Inventor: Homayoun Sadeghi, Durham, NC (US)

(21) Appl. No.: 13/141,049

(22) PCT Filed: Dec. 18, 2009
(86) PCT No.: PCT/US09/68656

§ 371 Date: Jul. 28, 2011

(30) Foreign Application Priority Data
Dec. 18, 2008 (US) 61138760
Feb. 13, 2009 (US) 61152504

(51) Int. Cl. A61K 38/17; A61K 38/22; C07K 14/575; C12P 21/00; C07K 14/00

(52) U.S. Cl. 514/1.1; 435/69.1; 530/350; 530/399; 530/324

(57) ABSTRACT

The present invention provides biologically active proteins that are activatable by peptidase exposure, such as dipeptidase exposure. The biologically active protein may be a recombinant version of a protein factor that is processed from a native precursor molecule in vivo. Upon administration of the recombinant product to a patient in need, the proteins are converted to the active form in the body by endogenous dipeptidase. The design of such products simplifies the manufacturing process, and may provide for additional therapeutic benefits such as improved pharmacokinetics, half-life, and/or safety profile. The present invention further provides methods of treatment with such compounds, as well as methods of production and/or manufacture.

### CROSS-REFERENCE TO RELATED APPLICATIONS

This application claims priority to U.S. Provisional Application Ser. No. 61/138,760, filed Dec. 18, 2008, and U.S. Provisional Application Ser. No. 61/152,504, filed Feb. 13, 2009, each of which is hereby incorporated by reference in its entirety for all purposes.

### FIELD OF THE INVENTION

The present invention relates to protein biologics and their use for the treatment of various medical conditions in human or veterinary patients. Particularly, the present invention relates to proteins whose biological activity is activatable by peptidase processing, including endogenous dipeptidase processing.

### BACKGROUND

The manufacture of recombinant protein biologics in their active forms can be a complicated, expensive, and non-optimal procedure. For example, some biologics are active, or fully active, only with the natural N-terminus of the protein factor. In nature, such biologically active proteins may result from precursor processing in the body, and this processing is difficult and cost intensive to recreate in the recombinant manufacturing process, especially on a large scale. For example, GLP-1, a potent physiological incretin, is a 37-amino acid peptide originating from the processing of preproglucagon. Processing of preproglucagon gives GLP-1 (7-36)amide and GLP-1 (7-37), for example, where the histidine at position 7 of the precursor molecule is at the N-terminus of the active molecule. Thus, during recombinant production of GLP-1, the N-terminal methionine necessary to initiate translation of the recombinant molecule must be removed to expose the N-terminal His, thereby producing the active drug.

### SUMMARY OF THE INVENTION

The present invention provides protein biologics that are activatable by peptidase or protease processing, including processing by endogenous peptidase (e.g., dipeptidase) or protease. For example, upon administration to a patient in need, the protein may be converted to the active form (or a more active form) in the body. The design of such recombinant products simplifies the manufacturing process, and may provide for additional therapeutic benefits such as improved pharmacokinetics, half-life (e.g., stability), and/or safety profile. The present invention further provides methods of treatment with such compounds, as well as methods of production and/or manufacture.

In one aspect, the invention provides a protein having a biological activity that is activatable by protease (e.g. a peptidase) processing. The protein may be administered as a pharmaceutical composition, with one or more pharmaceutically-acceptable carriers, diluents, and/or excipients. Upon administration, the protein becomes active, or increases in activity, upon peptidase action. In some embodiments, the protein is designed to be processed or activated by a dipeptidyl peptidase, such as DPP-IV. The biologically active protein may be GLP1, GLP2, glucagon, Exendin, Vasoactive Intestinal Peptide (VIP) or other protein or peptide drug. The biologically active protein may have additional components to improve therapeutic properties of the molecule, such as fusions of elastin-like protein (ELP), transferrin, albumin, or antibody sequences.

In another aspect, the invention provides methods of treatment for various conditions or disorders in human or veterinary patients. The method comprises administering the activatable protein of the invention to a patient in need. In accordance with this aspect, the activatable protein may provide for improved therapeutic performance of the therapeutic protein, including improved pharmacokinetics, stability, and safety profile, for example. In certain embodiments, the invention provides an activatable GLP1 molecule, optionally having a C-terminal ELP fusion, for use in treating diabetic patients among others.

In still other aspects, the invention provides methods for the production or manufacture of recombinant protein therapeutics, including recombinant therapeutics that mimic natural products produced by proteolytic processing in vivo. Such products in accordance with the invention are produced as recombinant proteins, so as to be activatable in vivo or in vitro by a peptidase or protease, such as DPP-IV or other dipeptidase. In certain embodiments, the recombinant protein therapeutic is manufactured with a non-natural N-terminus that is a substrate for a peptidase (e.g., a dipeptidase), and the recombinant protein therapeutic is not subjected to ex vivo manufacturing steps to create or expose the natural N-terminus (e.g., by in vitro peptidase processing). Such molecules are instead processed in vivo by endogenous factors upon administration to the patient.

### DESCRIPTION OF THE FIGURES

FIG. 1 illustrates an exemplary activatable GLP1 protein of the invention.

FIG. 1A is a GLP1 containing Ala-Ala at the N-terminus (bold type), which is removed by dipeptidase processing to expose the natural N-terminal His of GLP1(7-37). The molecule further contains a substitution of Gly at position 8 (position 2 with respect to N-terminal His), to prevent unwanted proteolysis. The exemplary molecule further comprises an ELP fusion at the C-terminus to extend half-life. The ELP fusion sequence, designated as ELP1-120, comprises 12 repeats of an ELP1 motif (VPGXG)₁₀, where X=V₅G₃A₂. FIG. 1B illustrates the same molecule after dipeptidase processing, having the His⁷ of GLP1(7-37) as the N-terminus.

FIG. 2 shows the results of a cAMP production assay by CHO cells containing human GLP1 receptor. These cells respond to the increasing concentrations of GLP1 and its active analogues x-axis) by producing cAMP. PB0967 (designated 967 on the graph) is a GLP1-ELP construct with Met-Ala-Ala at the N-terminus. It is anticipated that the Met is removed by E. coli during expression. As shown, the protein (∘) is inactive, and is activated by treatment with DPP-IV (♦), which removes the N-terminal Ala-Ala to expose the N-terminal His required for GLP1 activity.

FIG. 3 shows the results of a cAMP assay comparing two GLP1 constructs with MAA and MSP at the N-terminus before His⁷, respectively. FIG. 3 shows the results with protein treated with rDPP-IV, untreated protein, and with PB0868 (GLP1-ELP1-90). For comparison, in this assay, Exendin-4 peptide has an EC50 of around 1 nM.

FIG. 4 shows Intraperitoneal Glucose Tolerance Testing (IPGTT) in normal mice 12 hours after injection of PB967 (dose was about 30 nmol/Kg) (see FIG. 1) or buffer. The results demonstrate that injection of PB967 provides reduction in glucose excursion and rapid recovery to baseline. PB967 was therefore processed in vivo to the active form.

FIG. 5 shows blood pressure changes in Spontaneously Hypertensive (SH) rats injected subcutaneously with 10 mg/kg of maa VIP-ELP (PB1047) or buffer (control). The results demonstrate that PB1047 treated animals showed a significant difference in blood pressure at 4 hours post injection both in their systolic and diastolic pressure compared to controls.

### DETAILED DESCRIPTION OF THE INVENTION

The present invention provides recombinant protein products that are activatable by peptidase or protease processing. Thus, in certain embodiments, the protein is inactive or substantially inactive due to a masking of the active protein's N-terminus by one or more amino acid residues. These one or more amino acids are a substrate for a peptidase, such as a dipeptidase, so as to expose the N-terminus required for activity (or for higher activity) upon exposure to the peptidase in vitro or in vivo. For example, upon administration to a patient in need, the protein is converted to an active form in the body by endogenous protease or peptidase action. The design of such proteins simplifies the manufacturing process, and may provide for additional therapeutic benefits such as improved pharmacokinetics, half-life (e.g., stability), and/or safety profile. The present invention further provides methods of treatment with such products, as well as methods of production and/or manufacture.

### Activatable Proteins

In one aspect, the invention provides a therapeutic protein that is activatable by peptidase processing. The protein may be administered as a pharmaceutical composition as described herein, with one or more pharmaceutically-acceptable carriers, diluents, and/or excipients. Upon administration to a human or veterinary patient, the protein is activated by endogenous peptidase action.

In certain embodiments, the biologically active protein is a recombinant version of a protein factor that is processed from a native precursor molecule. Such molecules include a number of known peptide hormones, chemokines, neuropeptides, and vasoactive peptides. An exemplary biologically active molecule is GLP1 or other molecule that results from the processing of preproglucagon. For example, the biologically active protein may require an N-terminal amino acid other than methionine for activity, or for full activity. In some embodiments, the N-terminal amino acid of the biologically active protein (e.g., the N-terminal amino acid required for activity) is His or other amino acid that prohibits, restricts, or limits the removal of the N-terminal methionine by E. coli or other expression system. Thus, in certain embodiments, the N-terminal amino acid required for activity is not Gly, Ala, Ser, Cys, Thr, Val, or Pro, as each of which will trigger the removal of an N-terminal methionine in E. coli.

Exemplary biologically active proteins that find use with the invention include GLP1, GLP2, glucagon, Growth Hormone-Releasing Factor (GRF), insulin, and Vasoactive Intestinal Peptide (VIP). Other biologically active peptides include those described in Table 1 of U.S. Provisional Application No. 61/106,476 filed Oct. 17, 2008, which is hereby incorporated by reference in its entirety. The native and recombinant amino acid sequences of such peptides are disclosed in U.S. Application No. 61/106,476, and/or are known in the art, and such sequences are hereby incorporated by reference. Such proteins, designed to be activated in accordance with the invention, may be administered for the treatment of a condition or disease listed in Table 1 of U.S. Provisional Application No. 61/106,476. Certain exemplary biologically active proteins that may be employed in connection with the invention are described in greater detail herein.

The recombinant protein of the invention is designed to be processed or activated by a peptidase or protease, such as an endogenous peptidase or protease. As used herein, the terms “peptidase” and “protease” are interchangeable. For example, the prodrug may be designed to be activatable by a dipeptidyl peptidase. Exemplary dipeptidyl peptidases include dipeptidyl peptidase-1 (DPP-I), dipeptidyl peptidase-3 (DPP-III), dipeptidyl peptidase-4 (DPP-IV), dipeptidyl peptidase-6 (DPP-VI), dipeptidyl peptidase-7 (DPP-VII), dipeptidyl peptidase-8 (DPP-VIII), dipeptidyl peptidase-9 (DPP-IX), dipeptidyl peptidase-10 (DPP-X). Substrate sequences for such dipeptidases are known.

In certain embodiments, the prodrug is designed to be activatable by DPP-IV. DPP-IV is an enzyme expressed on the surface of most cell types and is associated with immune regulation, signal transduction and apoptosis. It is an intrinsic membrane glycoprotein and a serine exopeptidase that cleaves X-proline dipeptides from the N-terminus of polypeptides. Substrates of DPP-IV include proline or alanine-containing peptides, and include such endogenous molecules as growth factors, chemokines, neuropeptides, and vasoactive peptides. In fact, DPP-IV is responsible for the degradation of incretins such as the endogenous GLP1. A new class of oral hypoglycemics called dipeptidyl peptidase-4 inhibitors work by inhibiting the action of DPP-IV, thereby prolonging endogenous incretin effect in vivo. However, in accordance with these embodiments of the present invention, DPP-IV activity activates the therapeutic protein (e.g., GLP1), by removing an N-terminal DPP-IV-sensitive dipeptide. Thus, the invention takes advantage of the endogenous and ubiquitous DPP-IV activity that has previously been a hurdle to maintaining endogenous or exogenously-delivered incretin activity.

Thus, the recombinant proteins of the invention may be sensitive to a dipeptidase, such as DPP-IV. For example, the N-terminus of the protein may have the structure Z-N, where Z is a dipeptide substrate for dipeptidase (e.g., Z is removed by dipeptidase exposure), and N is the N-terminus of the biologically active molecule. In exemplary embodiments, the protein may have an N-terminal sequence with the formula M-X-N where M is methionine, X is Pro, Ala, or Ser, and N is the desired N-terminus of the biologically active molecule. In this manner, M-X will be sensitive to dipeptidase, such as DPP-IV. Alternatively, the N-terminal sequence of the protein may be X¹-X²—N, where X′ is Gly, Ala, Ser, Cys, Thr, Val, or Pro, and X² is Pro, Ala, or Ser. X¹-X² is a substrate for dipeptidase such as DPP-IV, and dipeptidase digestion will expose N, the desired N-terminus of the biologically active molecule. In such embodiments, the protein may be conveniently produced by expression of a construct encoding M-X¹-X²—N (where M is methionine) in E. coli, since Gly, Ala, Ser, Cys, Thr, Val, or Pro at the second position will signal the removal of the Met, thereby leaving X¹-X² on the N-terminus.

The biologically active protein or peptide may have additional components to improve therapeutic properties of the molecule, such as fusions with elastin-like protein (ELP), transferrin, albumin, or antibody sequences. Such sequences are known in the art for providing certain beneficial properties associated with stability and half-life, for example. See U.S. Pat. No. 7,238,667 (particularly with respect to albumin conjugates), U.S. Pat. No. 7,176,278 (particularly with respect to transferrin conjugates), U.S. Pat. No. 5,766,883, and WO 2008/030968 (with respect to ELP conjugates), which are each hereby incorporated by reference in their entireties.

### Glucagon-Like Peptide (GLP)-1 Receptor Agonists

In certain embodiments of the invention, the therapeutic agent is a GLP1 receptor agonist, such as GLP1, Exendin, or functional analogs thereof (which may contain ELP fusion sequences as described herein). In accordance with these embodiments, the GLP1 receptor agonist is initially inactive, but is activated by peptidase or protease exposure, such as a dipeptidyl peptidase (e.g., DPP-IV).

Human GLP-1 is a 37 amino acid residue peptide originating from preproglucagon which is synthesised in the L-cells in the distal ileum, in the pancreas, and in the brain. Processing of preproglucagon to give GLP-1 (7-36)amide, GLP-1 (7-37) and GLP-2 occurs mainly in the L-cells. A simple system is used to describe fragments and analogs of this peptide. For example, Gly⁸-GLP-1 (7-37) designates a fragment of GLP-1 formally derived from GLP-1 by deleting the amino acid residues Nos. 1 to 6 and substituting the naturally occurring amino acid residue in position 8 (Ala) by Gly. Similarly, Lys³⁴ (N^{ε}-tetradecanoyl)-GLP-1(7-37) designates GLP-1 (7-37) wherein the ε-amino group of the Lys residue in position 34 has been tetradecanoylated. Where reference is made to C-terminally extended GLP-1 analogues (other than C-terminal fusion sequences), the amino acid residue in position 38 is Arg unless otherwise indicated, the optional amino acid residue in position 39 is also Arg unless otherwise indicated and the optional amino acid residue in position 40 is Asp unless otherwise indicated. Also, if a C-terminally extended analogue extends to position 41, 42, 43, 44 or 45, the amino acid sequence of this extension is as in the corresponding sequence in human preproglucagon unless otherwise indicated.

The parent peptide of GLP-1, proglucagon (PG), has several cleavage sites that produce various peptide products dependent on the tissue of origin including glucagon (PG[32-62]) and GLP-1[7-36]NH₂ (PG[72-107]) in the pancreas, and GLP-1[7-37] (PG[78-108]) and GLP-1[7-36]NH₂ (PG[78-107]) in the L cells of the intestine where GLP-1 [7-36]NH₂ (78-107 PG) is the major product. The GLP-1 component in accordance with the invention may be any biologically active product or deivative of proglocagon, or functional analog thereof, including: GLP-1 (1-35), GLP-1 (1-36), GLP-1 (1-36)amide, GLP-1 (1-37), GLP-1 (1-38), GLP-1 (1-39), GLP-1 (1-40), GLP-1 (1-41), GLP-1 (7-35), GLP-1 (7-36), GLP-1 (7-36)amide, GLP-1 (7-37), GLP-1 (7-38), GLP-1 (7-39), GLP-1 (7-40) and GLP-1 (7-41), or a analog of the foregoing. Generally, the GLP-1 component in some embodiments may be expressed as GLP-1 (A-B), where A is an integer from 1 to 7 and B is an integer from 38 to 45, optionally with one or more amino acid substitutions as defined below. In various embodiments of the invention A is 7, which provides activatable GLP1 molecules when His⁷ is masked by a dipeptidyl-sensitive sequence.

As an overview, after processing in the intestinal L-cells, GLP-1 is released into the circulation, most notably in response to a meal. The plasma concentration of GLP-1 rises from a fasting level of approximately 15 pmol/L to a peak postprandial level of 40 pmol/L. For a given rise in plasma glucose concentration, the increase in plasma insulin is approximately threefold greater when glucose is administered orally compared with intravenously (Kreymann et al., 1987, Lancet 2(8571): 1300-4). This alimentary enhancement of insulin release, known as the incretin effect, is primarily humoral and GLP-1 is now thought to be the most potent physiological incretin in humans. GLP-1 mediates insulin production via binding to the GLP-1 receptor, known to be expressed in pancreatic 13 cells. In addition to the insulinotropic effect, GLP-1 suppresses glucagon secretion, delays gastric emptying (Wettergen et al., 1993, Dig Dis Sci 38: 665-73) and may enhance peripheral glucose disposal (D'Alessio et al., 1994, J. Clin Invest 93: 2293-6).

A combination of actions gives GLP-1 unique therapeutic advantages over other agents currently used to treat non-insulin-dependent diabetes mellitus (NIDDM). First, a single subcutaneous dose of GLP-1 can completely normalize post prandial glucose level's in patients with NIDDM (Gutniak et al., 1994, Diabetes Care 17: 1039-44). This effect may be mediated both by increased insulin release and by a reduction in glucagon secretion. Second, intravenous infusion of GLP-1 can delay postprandial gastric emptying in patients with NIDDM (Williams et al., 1996, J. Clin Endo Metab 81: 327-32). Third, unlike sulphonylureas, the insulinotropic action of GLP-1 is dependent on plasma glucose concentration (Holz et al., 1993, Nature 361:362-5). Thus, the loss of GLP-1-mediated insulin release at low plasma glucose concentration protects against severe hypoglycemia.

When given to healthy subjects, GLP-1 potently influences glycemic levels as well as insulin and glucagon concentrations (Orskov, 1992, Diabetologia 35:701-11), effects which are glucose dependent (Weir et al., 1989, Diabetes 38: 338-342). Moreover, it is also effective in patients with diabetes (Gutniak, M., 1992, N. Engl J Med 226: 1316-22), normalizing blood glucose levels in type 2 diabetic subjects and improving glycemic control in type 1 patients (Nauck et al., 1993, Diabetologia 36: 741-4, Creutzfeldt et al., 1996, Diabetes Care 19:580-6).

GLP-1 is, however, metabolically unstable, having a plasma half-life (t_{1/2}) of only 1-2 minutes in vivo. Moreover, exogenously administered GLP-1 is also rapidly degraded (Deacon et al., 1995, Diabetes 44: 1126-31). This metabolic instability has limited the therapeutic potential of native GLP-1.

GLP-1 [7-37] has the following amino acid sequence: HAEGTFTSDVSSYLEGQAAKEFIAWLVKGRG (SEQ ID NO:13), which may be employed as the GLP-1 component in the activatable protein of the invention. That is, the protein comprises SEQ ID NO:13 with an activatable sequence at the N-terminus as described, to expose the N-terminus of SEQ ID NO:13 in vivo. Alternatively, the GLP-1 component may contain glycine (G) at the second position, giving, for example, the activated sequence of HGEGTFTSDVSSYLEGQAAKEFIAWLVKGRG (SEQ ID NO:14), which may further comprise ELP or other fusion sequences at the C-terminus. The GLP-1 component may be a biologically active fragment of GLP-1, for example, as disclosed in US 2007/0041951, which is hereby incorporated by reference in its entirety. Other fragments and modified sequences of GLP-1 are known in the art (U.S. Pat. No. 5,614,492; U.S. Pat. No. 5,545,618; European Patent Application, Publication No. EP 0658568 A1; WO 93/25579, which are hereby incorporated by reference in their entireties). Such fragments and modified sequences may be used in connection with the present invention, as well as those described below.

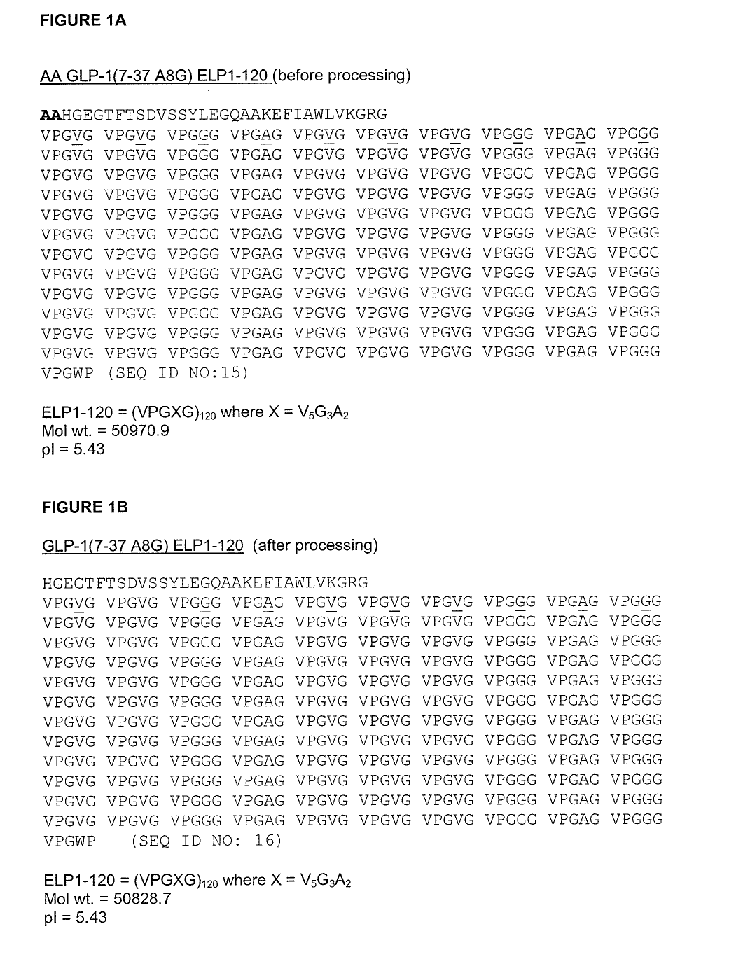

For example, the N-terminus of the activatable GLP1 may have the structure Z-N, where Z is a substrate for a dipeptidase (e.g., Z is removed by dipeptidase exposure), and N is His⁷ of GLP1, the N-terminus desired for biological activity. The activatable GLP1 may have an N-terminal sequence with the formula M-X-N where M is methionine, X is Pro, Ala, or Ser, and N is His⁷ of GLP1. In this manner, M-X will be sensitive to, and removed by, dipeptidase such as DPP-IV. Alternatively, the N-terminal sequence of the activatable GLP1 may be X¹-X²-N, where X¹ is Gly, Ala, Ser, Cys, Thr, Val, or Pro; X² is Pro, Ala, or Ser; and N is His⁷ of GLP1. X¹-X² is a substrate for dipeptidase such as DPP-IV, and dipeptidase digestion will expose N, the desired N-terminus of the biologically active molecule (See SEQ ID NO:15 illustrated in FIG. 1. In such embodiments, the protein may be produced by expression of a construct encoding M-X¹-X²-N (where M is methionine) in E. coli, since Gly, Ala, Ser, Cys, Thr, Val, or Pro at the second position will signal the removal of the Met, thereby leaving X¹-X² on the N-terminus. See SEQ ID NO:16 in FIG. 1.

Certain structural and functional analogs of GLP-1 have been isolated from the venom of the Gila monster lizards (Heloderma suspectum and Heloderma horridum) and have shown clinical utility. Such molecules find use in accordance with the present invention. In particular, exendin-4 is a 39 amino acid residue peptide isolated from the venom of Heloderma suspectum and shares approximately 52% homology with human GLP-1. Exendin-4 is a potent GLP-1 receptor agonist that stimulates insulin release, thereby lowering blood glucose levels. Exendin-4 has the following amino acid sequence: HGEGTFTSDLSKQMEEEAVRLFIEWLKNGGPSSGAPPPS (SEQ ID NO:17). A synthetic version of exendin-4 known as exenatide (marketed as Byetta®) has been approved for the treatment of Type-2 Diabetes. Although exenatide is structurally analogous to native GLP-1, it has a longer half-life after injection. Exendin-4 may be designed as an activatable peptide in connection with Z-N, M-X-N, and X¹-X²-N constructs as described.

While exenatide has the ability to lower blood glucose levels on its own, it can also be combined with other medications such as metformin, a thiozolidinedione, a sulfonylureas, and/or insulin to improve glucose control. Exenatide is administered by injection subcutaneously twice per day using a pre-filled pen device. Typical human responses to exenatide include improvements in the initial rapid release of endogenous insulin, an increase in β-cell growth and replication, suppression of pancreatic glucagon release, delayed gastric emptying, and reduced appetite—all of which function to lower blood glucose. Unlike sulfonylureas and meglitinides, exenatide increases insulin synthesis and secretion in the presence of glucose only, thus lessening the risk of hypoglycemia. Despite the therapeutic utility of exenatide, it has certain undesirable traits, including the requirement of twice daily injections, gastrointestional side effects, and similar to native GLP-1, a relatively short half-life (i.e. approximately 2 hr).

Various functional analogs of GLP-1 and exendin-4 are known, and which find use in accordance with the invention. These include liraglutide (Novo Nordisk, WO98/008871), R1583/taspoglutide (Roche, WO00/034331), CJC-1131 (ConjuChem, WO00/069911), ZP-10/AVE0010 (Zealand Pharma, Sanofi-Aventis, WO01/004156), and LY548806 (Eli Lilly, WO03/018516).

Liraglutide, also known as NN2211, is a GLP-1 receptor agonist analog that has been designed for once-daily injection (Harder et al., 2004, Diabetes Care 27: 1915-21). Liraglutide has been tested in patients with type-2 diabetes in a number of studies and has been shown to be effective over a variety of durations. In one study, treatment with liraglutide improved glycemic control, improved β-cell function, and reduced endogenous glucose release in patients with type-2 diabetes after one week of treatment (Degn et al., 2004, Diabetes 53: 1187-94). In a similar study, eight weeks of 0.6-mg liraglutide therapy significantly improved glycemic control without increasing weight in subjects with type 2 diabetes compared with those on placebo (Harder et al., 2004, Diabetes Care 27: 1915-21).

Thus, in certain embodiments, the GLP-1 receptor agonist in accordance with the invention is as described in WO98/008871, which is hereby incorporated by reference in its entirety. The GLP-1 receptor agonist may have at least one lipophilic substituent, in addition to one, two, or more amino acid substitutions with respect to native GLP-1. For example, the lipophilic substituent may be an acyl group selected from CH₃(CH₂)ₙCO—, wherein n is an integer from 4 to 38, such as an integer from 4 to 24. The lipophilic substituent may be an acyl group of a straight-chain or branched alkyl or fatty acid (for example, as described in WO98/008871, which description is hereby incorporated by reference).

In certain embodiments, the GLP-1 component is Arg²⁶-GLP-1 (7-37), Arg³⁴-GLP-1(7-37), Lys³⁶-GLP-1 (7-37), Arg^{26,34}Lys³⁶-GLP-I (7-37), Arg^{26,34}Lys³⁸-GLP-I (7-38), Arg^{28,34} Lys³⁹-GLP-1 (7-39), Arg^{26,34}Lys⁴⁰-GLP-1(7-40), Arg²⁶Lys³⁶-GLP-1(7-37), Arg³⁴Lys³⁶-GLP-1(7-37), Arg²⁶Lys³⁹-GLP-1(7-39), Arg³⁴Lys⁴⁰-GLP-1(7-40), Arg^{26,34}Lys^{36,39}-GLP-I (7-39), Arg^{26,34}Lys^{36,40}-GLP-1(7-40), Gly⁸Arg²⁶-GLP-1 (7-37); Gly⁸Arg³⁴-GLP-1(7-37); Gly⁸Lys³⁸-GLP-26,34Lys⁴⁰-GLP-1(7-37); Gly⁸Arg^{26,34}Lys³⁶-GLP-1(7-37), Gly⁸Arg^{26,34}Lys³⁹-GLP-1(7-39), Gly⁸Arg^{26,34}Lys⁴⁰-GLP-1(7-40), Gly⁸Arg²⁶Lys³⁶-GLP-1(7-37), Gly⁸Arg³⁴Lys³⁶-GLP-1(7-37), Gly⁸Arg²⁶Lys³⁹-GLP-1(7-39); Gly⁸Arg³⁴Lys⁴⁰-GLP-1(7-40), Gly⁸Arg^{28,34}Lys^{36,39}-GLP-1(7-39) and Gly⁸Arg^{26,34}Lys^{35,40}-GLP-1(7-40), each optionally having a lipophilic substituent. For example, the GLP-1 receptor agonist may have the sequence/structure Arg³⁴Lys²⁶-(N-ε-(γ-Glu(N-α-hexadecanoyl)))-GLP-I(7-37).

Taspoglutide, also known as R1583 or BIM 51077, is a GLP-1 receptor agonist that has been shown to improve glycemic control and lower body weight in subjects with type 2 diabetes mellitus treated with metformin (Abstract No. A-1604, Jun. 7, 2008, 68th American Diabetes Association Meeting, San Francisco, Calif.).

Thus, in certain embodiments, the GLP-1 receptor agonist is as described in WO00/034331, which is hereby incorporated by reference in its entirety. In certain exemplary embodiments, the GLP-1 receptor agonist has the sequence [Aib^{8,35}]hGLP-1(7-36)NH₂ (e.g. taspoglutide), wherein Aib is alpha-aminoisobutyric acid.

CJC-1131 is a GLP-1 analog that consists of a DPP-IV-resistant form of GLP-1 joined to a reactive chemical linker group that allows GLP-1 to form a covalent and irreversible bond with serum albumin following subcutaneous injection (Kim et al., 2003, Diabetes 52: 751-9). In a 12-week, randomized, double-blind, placebo-controlled multicenter study, CJC-1131 and metformin treatment was effective in reducing fasting blood glucose levels in type 2 diabetes patients (Ratner et al., Abstract No. 10-OR, June 10-14, 2005, 65th American Diabetes Association Meeting, San Francisco, Calif.).

Thus, in certain embodiments, the GLP-1 receptor agonist is as described in WO00/069911, which is hereby incorporated by reference in its entirety. In some embodiments, the GLP-1 receptor agonist is modified with a reactive group which reacts with amino groups, hydroxyl groups or thiol groups on blood components to form a stable covalent bond. In certain embodiments, the GLP-1 receptor agonist is modified with a reactive group selected from the group consisting of succinimidyl and maleimido groups. In certain exemplary embodiments, the GLP-1 receptor agonist has the sequence/structure: D-Ala⁸Lys³⁷-(2-(2-(2-maleimidopropionamido(ethoxy)ethoxy)acetamide))-GLP-1(7-37) (e.g. CJC-1131).

AVE0010, also known as ZP-10, is a GLP-1 receptor agonist that may be employed in connection with the invention. In a recent double-blind study, patients treated with once daily dosing of AVE0010 demonstrated significant reductions in HbA1c levels (Ratner et al., Abstract No. 433-P, 68th American Diabetes Association Meeting, San Francisco, Calif.). At the conclusion of the study, the percentages of patients with HbA1c<7% ranged from 47-69% for once daily dosing compared to 32% for placebo. In addition, AVE0010 treated patients showed dose-dependent reductions in weight and post-prandial plasma glucose.

Thus, in certain embodiments, the GLP-1 receptor agonist is as described in WO01/004156, which is hereby incorporated by reference in its entirety. For example, the GLP-1 receptor agonist may have the sequence:

(SEQ ID NO: 18)
HGEGTFTSDLSKQMEEEAVRLFIEWLKNGGPSSGAPPSKKKKKK-NH2
(e.g. AVE0010).

LY548806 is a GLP-1 derivative designed to be resistant to proteolysis by dipeptidase-peptidyl IV (DPP-IV) (Jackson et al., Abstract No. 562, Jun. 10-14, 2005, 65th American Diabetes Association Meeting, San Francisco, Calif.). In an animal model of hyperglycemia, LY548806 has been shown to produce a significant lowering of blood glucose levels during the hyperglycemic phase (Saha et al., 2006, J. Pharm. Exp. Ther. 316: 1159-64). Moreover, LY548806 was shown to produce a significant increase in insulin levels consistent with its known mechanism of action, namely stimulation of insulin release in the presence of hyperglycemia.

Thus, in certain embodiments, the GLP-1 receptor agonist is as described in WO03/018516, which is hereby incorporated by reference in its entirety. In some embodiments, the therapeutic agents of the present invention comprise GLP-1 analogs wherein the backbone for such analogs or fragments contains an amino acid other than alanine at position 8 (position 8 analogs). The backbone may also include L-histidine, D-histidine, or modified forms of histidine such as desamino-histidine, 2-amino-histidine, β-hydroxy-histidine, homohistidine, α-fluoromethyl-histidine, or α-methyl-histidine at position 7. In some embodiments, these position 8 analogs may contain one or more additional changes at positions 12, 16, 18, 19, 20, 22, 25, 27, 30, 33, and 37 compared to the corresponding amino acid of native GLP-1. In other embodiments, these position 8 analogs may contain one or more additional changes at positions 16, 18, 22, 25 and 33 compared to the corresponding amino acid of native GLP-1. In certain exemplary embodiments, the GLP-1 receptor agonist has the sequence:

(SEQ ID NO: 19)
HVEGTFTSDVSSYLEEQAAKEFIAWLIKGRG-OH
(e.g. LY548806).

For example, in certain embodiments, the GLP-1 receptor agonist is GLP-1 (e.g., SEQ ID NO:13 or 14) or a functional analog thereof. In other embodiments, the GLP-1 receptor agonist is exendin-4 (SEQ ID NO:17) or a functional analog thereof. Such functional analogs of GLP-1 or exendin-4 include functional fragments truncated at the C-terminus by from 1 to 10 amino acids, including by 1, 2, 3, or up to about 5 amino acids (with respect to SEQ ID NOS:13, 14, or 17). Such functional analogs may contain from 1 to 10 amino acid insertions, deletions, and/or substitutions (collectively) with respect to the native sequence (e.g., SEQ ID NOS:13, 14, or 17), and in each case retaining the activity of the peptide. For example, the functional analog of GLP-1 or exendin-4 may have from 1 to about 3, 4, or 5 insertions, deletions and/or substitutions (collectively) with respect to SEQ ID NOS:13, 14, and 17 (respectively), and in each case retaining the activity of the peptide. Such activity may be confirmed or assayed using any available assay, including those described herein. In these or other embodiments, the GLP-1 receptor agonist component has at least about 50%, 75%, 80%, 85%, 90%, or 95% identity with the native sequence (SEQ ID NOS: 13, 14 or 17). The determination of sequence identity between two sequences (e.g., between a native sequence and a functional analog) can be accomplished using any alignment tool, including Tatusova et al., Blast 2 sequences—a new tool for comparing protein and nucleotide sequences, FEMS Microbiol Lett. 174:247-250 (1999). Such functional analogs may further comprise additional chemical modifications, such as those described in this section and/or others known in the art.

The activatable GLP1 of the invention may be provided as a pharmaceutical composition, comprising one or more pharmaceutically-acceptable carriers, diluents, and/or excipients (as discussed in greater detail herein). The composition may comprise the protein in any pharmaceutically-acceptable form, including as a pharmaceutcally-acceptable salt. The composition may be formulated for administration by any suitable route, which may include administration by injection (e.g. subcutaneous injection). Suitable components and/or forms of such compositions are described in U.S. Provisional Application No. 61/106,476, which is hereby incorporated by reference in its entirety.

Pharmaceutical compositions in accordance with these embodiments (e.g., activatable GLP-1 receptor agonist fusions) may be dosed at from 1 mg to about 20 mg of active agent, e.g., for daily treatment. In some embodiments, the compositions may be dosed at from 5 mg to 10 mg of active agent for daily treatment. The compositions may be dosed at from about 15 mg to about 75 mg of active agent, e.g., for treatment every other day or bi-weekly. In some embodiments, the compositions are dosed at about 20 mg to about 150 mg of active agent for weekly treatment. For example, the compositions may be dosed at from about 40 mg to about 100 mg, or about 50 to 80 mg of active agent for weekly treatment. Thus, patient's may receive treatment daily, every other day, every third day, or weekly. The compositions, whether for daily, weekly, or bi-weekly treatment, may be formulated for administration by injection (e.g., subcutaneous injection). The compositions may be supplied in a pre-dosed form, e.g., pre-filled syringes, pens, or the like.

In another aspect, the present invention provides methods for the treatment or prevention of type 2 diabetes, impaired glucose tolerance, type 1 diabetes, hyperglycemia, obesity, binge eating, bulimia, hypertension, syndrome X, dyslipidemia, cognitive disorders, atheroschlerosis, non-fatty liver disease, myocardial infarction, coronary heart disease and other cardiovascular disorders. The method comprises administering the activatable GLP1 receptor agonist as described above to a patient in need of such treatment (e.g., a pharmaceutical composition as described above). In these or other embodiments, the present invention provides methods for decreasing food intake, decreasing β-cell apoptosis, increasing β-cell function and β-cell mass, and/or for restoring glucose sensitivity to β-cells. Generally, the patient may be a human or non-human animal patient (e.g., dog, cat, cow, or horse).

The treatment with the activatable GLP1 receptor agonist according to the present invention may also be combined with one or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. In the present context, the expression “antidiabetic agent” includes compounds for the treatment and/or prophylaxis of insulin resistance and diseases wherein insulin resistance is the pathophysiological mechanism.

The ability of a GLP-1 or exendin-4 analog, or an ELP/GLP-1 receptor agonist compound, to bind the GLP-1 receptor may be determined by standard methods, for example, by receptor-binding activity screening procedures which involve providing appropriate cells that express the GLP-1 receptor on their surface, for example, insulinoma cell lines such as RINmSF cells or INS-1 cells. In addition to measuring specific binding of tracer to membrane using radioimmunoassay methods, cAMP activity or glucose dependent insulin production can also be measured. In one method, a polynucleotide encoding the GLP-1 receptor is employed to transfect cells to thereby express the GLP-1 receptor protein. Thus, these methods may be employed for testing or confirming whether a suspected GLP-1 receptor agonist is active.

In addition, known methods can be used to measure or predict the level of biologically activity of a GLP-1 receptor agonist or ELP/GLP-1 receptor agonist in vivo (See e.g. Siegel, et al., 1999, Regul Pept 79(2-3): 93-102). In particular, GLP-1 receptor agonists or ELP/GLP-1 receptor agonist compounds can be assessed for their ability to induce the production of insulin in vivo using a variety of known assays for measuring GLP-1 activity. For example, an ELP/GLP-1 receptor agonist compound can be introduced into a cell, such as an immortalized β-cell, and the resulting cell can be contacted with glucose. If the cell produces insulin in response to the glucose, then the modified GLP-1 is generally considered biologically active in vivo (Fehmann et al., 1992, Endocrinology 130: 159-166).

The ability of an ELP/GLP-1 receptor agonist compound to enhance β-cell proliferation, inhibit β-cell apoptosis, and regulate islet growth may also be measured using known assays. Pancreatic β-cell proliferation may be assessed by ³H-tymidine or BrdU incorporation assays (See e.g. Buteau et al., 2003, Diabetes 52: 124-32), wherein pancreatic β-cells such as INS(832/13) cells are contacted with an ELP/GLP-1 receptor agonist compound and analyzed for increases in ³H-thymidine or BrdU incorporation. The antiapoptotic activity of an ELP/GLP-1 receptor agonist compound can be measured in cultured insulin-secreting cells and/or in animal models where diabetes occurs as a consequence of an excessive rate of beta-cell apoptosis (See e.g. Bulotta et al., 2004, Cell Biochem Biophys 40(3 suppl): 65-78).

In addition to GLP-1, other peptides of the family, such as those derived from processing of the pro-glucagon gene, such as GLP2, GIP, and oxyntomodulin, could be designed as activatable proteins in accordance with the present disclosure.

### Vasoactive Intestinal Peptide

In some embodiments, the therapeutic agent comprises an activatable vasoactive intestinal peptide (VIP), or functional analog thereof, and optionally a fusion component (such as an ELP component as described). VIP is a peptide hormone containing 28 amino acid residues and is produced in many areas of the human body including the gut, pancreas and suprachiasmatic nuclei of the hypothalamus in the brain. The unfused peptide has a half-life in the blood of about two minutes.

VIP has an effect on several parts of the body. With respect to the digestive system, VIP may induce smooth muscle relaxation (lower esophageal sphincter, stomach, gallbladder), stimulate secretion of water into pancreatic juice and bile, and cause inhibition of gastric acid secretion and absorption from the intestinal lumen. Its role in the intestine is to stimulate secretion of water and electrolytes, as well as dilating intestinal smooth muscle, dilating peripheral blood vessels, stimulating pancreatic bicarbonate secretion, and inhibiting gastrin-stimulated gastric acid secretion. These effects work together to increase motility. VIP has the function of stimulating pepsinogen secretion by chief cells.

VIP has been found in the brain and some autonomic nerves. One region of the brain includes a specific area of the suprachiasmatic nuclei (SCN), the location of the ‘master circadian pacemaker’. The SCN coordinates daily timekeeping in the body and VIP plays a key role in communication between individual brain cells within this region. Further, VIP is also involved in synchronising the timing of SCN function with the environmental light-dark cycle. Combined, these roles in the SCN make VIP a crucial component of the mammalian circadian timekeeping machinery.

VIP may help to regulate prolactin secretion.

VIP has been found in the heart and has significant effects on the cardiovascular system. It causes coronary vasodilation, as well as having a positive inotropic and chronotropic effect.

VIP has further been described as an immunomodulating peptide useful for treating inflammation and TH1-type autoimmune disease (See Delgado et al., The Significance of Vasoactive Intestinal Peptide in Immunomodulation, Pharmacol. Reviews 56(2):249-290 (2004)). VIP has been further been described as useful for the treatment of neurodegenerative diseases (see U.S. Pat. No. 5,972,883, which is hereby incorporated by reference in its entirety).

VIP is a 28 amino acid peptide having the following amino acid sequence: HSDAVFTDNYTRLRKQMAVKKYLNSILN (SEQ ID NO: 20). VIP results from processing of the 170-amino acid precursor molecule prepro-VIP. Structures of VIP and analogs have been described in U.S. Pat. Nos. 4,734,487, 4,737,487, 4,835,252, 4,939,224, and 6,489,297, each of which is hereby incorporated by reference in its entirety.

Thus, in certain embodiments, the activatable protein is an activatable VIP (e.g., comprising SEQ ID NO:20) or a functional analog thereof. Such functional analogs of VIP include functional fragments truncated at the N- or C-terminus by from 1 to 10 amino acids, including by 1, 2, 3, or up to about 5 amino acids (with respect to SEQ ID NO:20). Such functional analogs may contain from 1 to 5 amino acid insertions, deletions, and/or substitutions (collectively) with respect to the native sequence (e.g., SEQ ID NO:20), and in each case retaining the activity of the peptide, and particularly immunomodulating activity. Such activity may be confirmed or assayed using any available assay, including any suitable assay to determine or quantify an activity described in Delgado et al., The Significance of Vasoactive Intestinal Peptide in Immunomodulation, Pharmacol. Reviews 56(2):249-290 (2004). In these or other embodiments, the VIP component has at least about 50%, 75%, 80%, 85%, 90%, or 95% identity with the native sequence (SEQ ID NO:20). The determination of sequence identity between two sequences (e.g., between a native sequence and a functional analog) can be accomplished using any alignment tool, including Tatusova et al., Blast 2 sequences—a new tool for comparing protein and nucleotide sequences, FEMS Microbiol Lett. 174:247-250 (1999). Such functional analogs may further comprise additional chemical modifications, such as those described herein and/or others known in the art.

The N-terminus of the activatable VIP may have the structure Z-N, where Z is a substrate for a dipeptidase (e.g., Z is removed by dipeptidase exposure), and N is the N-terminal His of VIP. The activatable VIP may have an N-terminal sequence with the formula M-X-N where M is methionine, X is Pro, Ala, or Ser, and N is the N-terminal His of VIP. In this manner, M-X will be sensitive to, and removed by, dipeptidase such as DPP-IV. Alternatively, the N-terminal sequence of the activatable VIP may be X¹-X²-N, where X¹ is Gly, Ala, Ser, Cys, Thr, Val, or Pro; X² is Pro, Ala, or Ser; and N is the N-terminal His of VIP. X¹-X² is a substrate for dipeptidase such as DPP-IV, and dipeptidase digestion will expose N, the desired N-terminus of the biologically active molecule. In such embodiments, the protein may be produced by expression of a construct encoding M-X¹-X²-N (where M is methionine) in E. coli, since Gly, Ala, Ser, Cys, Thr, Val, or Pro at the second position will signal the removal of the Met, thereby leaving X¹-X² on the N-terminus.

The compositions of these embodiments, activatable VIP proteins optionally having fusion sequences (e.g., ELP fusion sequences), may be useful for the treatment of, among other things, cardiovascular disease, septic shock, rheumatoid arthritis, Crohn's Disease, Parkinson's Disease, and brain trauma. For example, the protein may be administered to a patient having such a condition, such that the peptide is activated in vivo to produce an effective amount of active VIP protein.

### Elastin-Like Protein Fusions

In certain embodiments, the protein product contains an ELP fusion at the C-terminus. The ELP component comprises or consists of structural peptide units or sequences that are related to, or derived from, the elastin protein. Such sequences are useful for improving the properties of therapeutic proteins in one or more of bioavailability, therapeutically effective dose and/or administration frequency, biological action, formulation compatibility, resistance to proteolysis, solubility, half-life or other measure of persistence in the body subsequent to administration, and/or rate of clearance from the body. See, for example, WO 2008/030968 which is hereby incorporated by reference in its entirety.

The ELP component is constructed from structural units of from three to about twenty amino acids, or in some embodiments, from four to ten amino acids, such as five or six amino acids. The length of the individual structural units, in a particular ELP component, may vary or may be uniform. In certain embodiments, the ELP component is constructed of a polytetra-, polypenta-, polyhexa-, polyhepta-, polyocta, and polynonapeptide motif of repeating structural units. Exemplary structural units include units defined by SEQ ID NOS: 1-12 (below), which may be employed as repeating structural units, including tandem-repeating units, or may be employed in some combination, to create an ELP effective for improving the properties of the therapeutic component. Thus, the ELP component may comprise or consist essentially of structural unit(s) selected from SEQ ID NOS: 1-12, as defined below.

The ELP component, comprising such structural units, may be of varying sizes. For example, the ELP component may comprise or consist essentially of from about 10 to about 500 structural units, or in certain embodiments about 20 to about 200 structural units, or in certain embodiments from about 50 to about 150 structural units, or from about 75 to about 130 structural units, including one or a combination of units defined by SEQ ID NOS: 1-12. The ELP component may comprise about 120 structural units, such as repeats of structural units defined by SEQ ID NO: 3 (defined below). Thus, the ELP component may have a length of from about 50 to about 2000 amino acid residues, or from about 100 to about 600 amino acid residues, or from about 200 to about 500 amino acid residues, or from about 200 to about 400 amino acid residues.

In some embodiments, the ELP component, or in some cases the therapeutic agent, has a size of less than about 150 kDa, or less than about 100 kDa, or less than about 55 kDa, or less than about 50 kDa, or less than about 40 kDa, or less than about 30 or 25 kDa.

In some embodiments, the ELP component in the untransitioned state may have an extended, relatively unstructured and non-globular form so as to escape kidney filtration. In such embodiments, the therapeutic agents of the invention have a molecular weight of less than the generally recognized cut-off for filtration through the kidney, such as less than about 60 kD, or in some embodiments less than about 55, 50, 45, 40, 30, or 25 kDa, and nevertheless persist in the body by at least 2-fold, 3-fold, 4-fold, 5-fold, 10-fold, 20-fold, or 100-fold longer than an uncoupled (e.g., unfused or unconjugated) therapeutic counterpart.

In these or other embodiments, the ELP component does not substantially or significantly impact the biological action of the therapeutic peptide. Thus, the (activated) therapeutic agent of the invention exhibits a potency (biological action) that is the same or similar to its unfused counterpart. The activated therapeutic agent of the invention may exhibit a potency or level of biological action (e.g., as tested in vitro or in vivo) of from 10-100% of that exhibited by the unfused counterpart of the therapeutic agent in the same assay. In various embodiments, the therapeutic agent may exhibit a potency or level of biological action (e.g., as tested in vitro or in vivo) of at least 50%, 60%, 75%, 80%, 90%, 95% or more of that exhibited by the unfused counterpart.

In certain embodiments, the ELP component undergoes a reversible inverse phase transition. That is, the ELP components are structurally disordered and highly soluble in water below a transition temperature (Tt), but exhibit a sharp (2-3° C. range) disorder-to-order phase transition when the temperature is raised above the Tt, leading to desolvation and aggregation of the ELP components. For example, the ELP forms insoluble polymers, when reaching sufficient size, which can be readily removed and isolated from solution by centrifugation. Such phase transition is reversible, and isolated insoluble ELPs can be completely resolubilized in buffer solution when the temperature is returned below the Tt of the ELPs. Thus, the therapeutic agents of the invention can, in some embodiments, be separated from other contaminating proteins to high purity using inverse transition cycling procedures, e.g., utilizing the temperature-dependent solubility of the therapeutic agent, or salt addition to the medium. Successive inverse phase transition cycles can be used to obtain a high degree of purity. In addition to temperature and ionic strength, other environmental variables useful for modulating the inverse transition of the therapeutic agents include pH, the addition of inorganic and organic solutes and solvents, side-chain ionization or chemical modification, and pressure.

In certain embodiments, the ELP component does not undergo a reversible inverse phase transition, or does not undergo such a transition at a biologically relevant Tt, and thus the improvements in the biological and/or physiological properties of the molecule (as described elsewhere herein), may be entirely or substantially independent of any phase transition properties. Nevertheless, such phase transition properties may impart additional practical advantages, for example, in relation to the recovery and purification of such molecules.

In certain embodiments, the ELP component(s) may be formed of structural units, including but not limited to:

(a) the tetrapeptide Val-Pro-Gly-Gly, or VPGG (SEQ ID NO: 1);

(b) the tetrapeptide Ile-Pro-Gly-Gly, or IPGG (SEQ ID NO: 2);

(c) the pentapeptide Val-Pro-Gly-X-Gly (SEQ ID NO: 3), or VPGXG, where X is any natural or non-natural amino acid residue, and where X optionally varies among polymeric or oligomeric repeats;

(d) the pentapeptide Ala-Val-Gly-Val-Pro, or AVGVP (SEQ ID NO: 4);

(e) the pentapeptide Ile-Pro-Gly-X-Gly, or IPGXG (SEQ ID NO: 5), where X is any natural or non-natural amino acid residue, and where X optionally varies among polymeric or oligomeric repeats;

(e) the pentapeptide Ile-Pro-Gly-Val-Gly, or IPGVG (SEQ ID NO: 6);

(f) the pentapeptide Leu-Pro-Gly-X-Gly, or LPGXG (SEQ ID NO: 7), where X is any natural or non-natural amino acid residue, and where X optionally varies among polymeric or oligomeric repeats;

(g) the pentapeptide Leu-Pro-Gly-Val-Gly, or LPGVG (SEQ ID NO: 8);

(h) the hexapeptide Val-Ala-Pro-Gly-Val-Gly, or VAPGVG (SEQ ID NO: 9);

(I) the octapeptide Gly-Val-Gly-Val-Pro-Gly-Val-Gly, or GVGVPGVG (SEQ ID NO: 10);

(J) the nonapeptide Val-Pro-Gly-Phe-Gly-Val-Gly-Ala-Gly, or VPGFGVGAG (SEQ ID NO: 11); and

(K) the nonapeptides Val-Pro-Gly-Val-Gly-Val-Pro-Gly-Gly, or VPGVGVPGG (SEQ ID NO: 12).

Such structural units defined by SEQ ID NOS: 1-12 may form structural repeat units, or may be used in combination to form an ELP component in accordance with the invention. In some embodiments, the ELP component is formed entirely (or almost entirely) of one or a combination of (e.g., 2, 3 or 4) structural units selected from SEQ ID NOS: 1-12. In other embodiments, at least 75%, or at least 80%, or at least 90% of the ELP component is formed from one or a combination of structural units selected from SEQ ID NOS: 1-12, and which may be present as repeating units.

In certain embodiments, the ELP component(s) contain repeat units, including tandem repeating units, of the pentapeptide Val-Pro-Gly-X-Gly (SEQ ID NO: 3), where X is as defined above, and where the percentage of Val-Pro-Gly-X-Gly (SEQ ID NO: 3) pentapeptide units taken with respect to the entire ELP component (which may comprise structural units other than VPGXG (SEQ ID NO: 3)) is greater than about 75%, or greater than about 85%, or greater than about 95% of the ELP component. The ELP component may contain motifs having a 5 to 15-unit repeat (e.g. about 10-unit or about 12-unit repeat) of the pentapeptide of SEQ ID NO: 3, with the guest residue X varying among at least 2 or at least 3 of the structural units within each repeat. The guest residues may be independently selected, such as from the amino acids V, I, L, A, G, and W (and may be selected so as to retain a desired inverse phase transition property). Exemplary motifs include VPGXG (SEQ ID NO: 3), where the guest residues are V (which may be present in from 40% to 60% of structural units), G (which may be present in 20% to 40% of structural units, and A (which may be present in 10% to 30% of structural units). The repeat motif itself may be repeated, for example, from about 5 to about 20 times, such as about 8 to 15 times (e.g., about 12 times), to create an exemplary ELP component. The ELP component as described in this paragraph may of course be constructed from any one of the structural units defined by SEQ ID NOS: 1-12, or a combination thereof. In exemplary ELP component is shown in FIG. 1 fused to the C-terminus of GLP1 [7-37].

In some embodiments, the ELP units may form a β-turn structure that provides an elastin-like property (e.g., inverse phase transition). Exemplary peptide sequences suitable for creating a β-turn structure are described in International Patent Application PCT/US96/05186, which is hereby incorporated by reference in its entirety. For example, the fourth residue (X) in the elastin pentapeptide sequence, VPGXG (SEQ ID NO:3), can be altered without eliminating the formation of a β-turn.

In certain embodiments, the ELP components include polymeric or oligomeric repeats of the pentapeptide VPGXG (SEQ ID NO: 3), where the guest residue X is any amino acid. X may be a naturally occurring or non-naturally occurring amino acid. In some embodiments, X is selected from alanine, arginine, asparagine, aspartic acid, cysteine, glutamic acid, glutamine, glycine, histidine, isoleucine, leucine, lysine, methionine, phenylalanine, serine, threonine, tryptophan, tyrosine and valine. In some embodiments, X is a natural amino acid other than proline or cysteine.

The guest residue X (e.g., with respect to SEQ ID NO: 3, or other ELP structural unit) may be a non-classical (non-genetically encoded) amino acid. Examples of non-classical amino acids include: D-isomers of the common amino acids, 2,4-diaminobutyric acid, α-amino isobutyric acid, A-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

Selection of X may be independent in each ELP structural unit (e.g., for each structural unit defined herein having a guest residue X). For example, X may be independently selected for each structural unit as an amino acid having a positively charged side chain, an amino acid having a negatively charged side chain, or an amino acid having a neutral side chain, including in some embodiments, a hydrophobic side chain.

In still other embodiments, the ELP component(s) may include polymeric or oligomeric repeats of the pentapeptides VPGXG (SEQ ID NO:3), IPGXG (SEQ ID NO:5) or LPGXG (SEQ ID NO:7), or a combination thereof, where X is as defined above.

In each embodiment, the structural units, or in some cases polymeric or oligomeric repeats, of the ELP sequences may be separated by one or more amino acid residues that do not eliminate the overall effect of the molecule, that is, in imparting certain improvements to the therapeutic component as described. In certain embodiments, such one or more amino acids also do not eliminate or substantially affect the phase transition properties of the ELP component (relative to the deletion of such one or more amino acids).

The structure of the resulting ELP components may be described using the notation ELPk [XᵢYᵢ-n], where k designates a particular ELP repeat unit, the bracketed capital letters are single letter amino acid codes and their corresponding subscripts designate the relative ratio of each guest residue X in the structural units (where applicable), and n describes the total length of the ELP in number of the structural repeats. For example, ELP1 [V₅A₂G₃-10] designates an ELP component containing 10 repeating units of the pentapeptide VPGXG (SEQ ID NO:3), where X is valine, alanine, and glycine at a relative ratio of 5:2:3; ELP1 [K₁V₂F₁-4] designates an ELP component containing 4 repeating units of the pentapeptide VPGXG (SEQ ID NO:3), where X is lysine, valine, and phenylalanine at a relative ratio of 1:2:1; ELP1 [K₁V₇F₁-9] designates a polypeptide containing 9 repeating units of the pentapeptide VPGXG (SEQ ID NO:3), where X is lysine, valine, and phenylalanine at a relative ratio of 1:7:1; ELP1 [V-5] designates a polypeptide containing 5 repeating units of the pentapeptide VPGXG (SEQ ID NO:3), where X is exclusively valine; ELP1 [V-20] designates a polypeptide containing 20 repeating units of the pentapeptide VPGXG (SEQ ID NO:3), where X is exclusively valine; ELP2 [5] designates a polypeptide containing 5 repeating units of the pentapeptide AVGVP (SEQ ID NO:4); ELP3 [V-5] designates a polypeptide containing 5 repeating units of the pentapeptide IPGXG (SEQ ID NO:5), where X is exclusively valine; ELP4 [V-5] designates a polypeptide containing 5 repeating units of the pentapeptide LPGXG (SEQ ID NO:7), where X is exclusively valine. Such ELP components as described in this paragraph may be used in connection with the present invention to increase the therapeutic properties of the therapeutic component.

Further, the Tt is a function of the hydrophobicity of the guest residue. Thus, by varying the identity of the guest residue(s) and their mole fraction(s), ELPs can be synthesized that exhibit an inverse transition over a 0-100° C. range. Thus, the Tt at a given ELP length may be decreased by incorporating a larger fraction of hydrophobic guest residues in the ELP sequence. Examples of suitable hydrophobic guest residues include valine, leucine, isoleucine, phenyalanine, tryptophan and methionine. Tyrosine, which is moderately hydrophobic, may also be used. Conversely, the Tt may be increased by incorporating residues, such as those selected from the group consisting of: glutamic acid, cysteine, lysine, aspartate, alanine, asparagine, serine, threonine, glycine, arginine, and glutamine; preferably selected from alanine, serine, threonine and glutamic acid.

The ELP component in some embodiments is selected or designed to provide a Tt ranging from about 10 to about 80° C., such as from about 35 to about 60° C., or from about 38 to about 45° C. In some embodiments, the Tt is greater than about 40° C. or greater than about 42° C., or greater than about 45° C., or greater than about 50° C. The transition temperature, in some embodiments, is above the body temperature of the subject or patient (e.g., >37° C.) thereby remaining soluble in vivo, or in other embodiments, the Tt is below the body temperature (e.g., <37° C.) to provide alternative advantages, such as in vivo formation of a drug depot for sustained release of the therapeutic agent. See, for example, US 2007/0009602, which is hereby incorporated by reference in its entirety.

The Tt of the ELP component can be modified by varying ELP chain length, as the Tt generally increases with decreasing MW. For polypeptides having a molecular weight>100,000, the hydrophobicity scale developed by Urry et al. (PCT/US96/05186, which is hereby incorporated by reference in its entirety) provides one means for predicting the approximate Tt of a specific ELP sequence. However, in some embodiments, ELP component length can be kept relatively small, while maintaining a target Tt, by incorporating a larger fraction of hydrophobic guest residues (e.g., amino acid residues having hydrophobic side chains) in the ELP sequence. For polypeptides having a molecular weight<100,000, the Tt may be predicted or determined by the following quadratic function: Tt=M₀ M₁X+M₂X² where X is the MW of the fusion protein, and M₀=116.21; M₁=−1.7499; M₂=0.010349.

While the Tt of the ELP component, and therefore of the ELP component coupled to a therapeutic component, is affected by the identity and hydrophobicity of the guest residue, X, additional properties of the molecule may also be affected. Such properties include, but are not limited to solubility, bioavailability, persistence, half-life, potency and safety of the molecule.

### Conjugation and Coupling

A recombinantly-produced ELP fusion protein, in accordance with certain embodiments of the invention, includes the ELP component and the therapeutic component associated with one another by genetic fusion. For example, the fusion protein may be generated by translation of a polynucleotide encoding the therapeutic component cloned in-frame with the ELP component.

In certain embodiments, the ELP component and the therapeutic components can be fused using a linker peptide of various lengths to provide greater physical separation and allow more spatial mobility between the fused portions, and thus maximize the accessibility of the therapeutic component, for instance, for binding to its cognate receptor. The linker peptide may consist of amino acids that are flexible or more rigid. For example, a flexible linker may include amino acids having relatively small side chains, and which may be hydrophilic. Without limitation, the flexible linker may contain a stretch of glycine and/or serine residues. More rigid linkers may contain, for example, more sterically hindering amino acid side chains, such as (without limitation) tyrosine or histidine. The linker may be less than about 50, 40, 30, 20, 10, or 5 amino acid residues. The linker can be covalently linked to and between an ELP component and a therapeutic component, for example, via recombinant fusion.

The linker or peptide spacer may be protease-cleavable or non-cleavable. By way of example, cleavable peptide spacers include, without limitation, a peptide sequence recognized by proteases (in vitro or in vivo) of varying type, such as Tev, thrombin, factor Xa, plasmin (blood proteases), metalloproteases, cathepsins (e.g., GFLG, etc.), and proteases found in other corporeal compartments. In some embodiments employing cleavable linkers, the fusion protein (“the therapeutic agent”) may be inactive, less active, or less potent as a fusion, which is then activated upon cleavage of the spacer in vivo. Alternatively, where the therapeutic agent is sufficiently active as a fusion, a non-cleavable spacer may be employed. The non-cleavable spacer may be of any suitable type, including, for example, non-cleavable spacer moieties having the formula [(Gly)n-Ser]m, where n is from 1 to 4, inclusive, and m is from 1 to 4, inclusive. Alternatively, a short ELP sequence different than the backbone ELP could be employed instead of a linker or spacer, while accomplishing the necessary effect.

In other embodiments, the present invention provides chemical conjugates of the ELP component and the activatable therapeutic component. The conjugates can be made by chemically coupling an ELP component to a therapeutic component by any number of methods well known in the art (See e.g. Nilsson et al., 2005, Ann Rev Biophys Bio Structure 34: 91-118). In some embodiments, the chemical conjugate can be formed by covalently linking the therapeutic component to the ELP component, directly or through a short or long linker moiety, through one or more functional groups on the therapeutic proteinacious component, e.g., amine, carboxyl, phenyl, thiol or hydroxyl groups, to form a covalent conjugate. Various conventional linkers can be used, e.g., diisocyanates, diisothiocyanates, carbodiimides, bis(hydroxysuccinimide) esters, maleimide-hydroxysuccinimide esters, glutaraldehyde and the like.

Non-peptide chemical spacers can additionally be of any suitable type, including for example, by functional linkers described in Bioconjugate Techniques, Greg T. Hermanson, published by Academic Press, Inc., 1995, and those specified in the Cross-Linking Reagents Technical Handbook, available from Pierce Biotechnology, Inc. (Rockford, Ill.), the disclosures of which are hereby incorporated by reference, in their respective entireties. Illustrative chemical spacers include homobifunctional linkers that can attach to amine groups of Lys, as well as heterobifunctional linkers that can attach to Cys at one terminus, and to Lys at the other terminus.

In certain embodiments, relatively small ELP components (e.g., ELP components of less than about 30 kDa, 25 kDa, 20 kDa, 15 kDa, or 10 kDa), that do not transition at room temperature (or human body temperature, e.g., Tt>37° C.), are chemically coupled or crosslinked. For example, two relatively small ELP components, having the same or different properties, may be chemically coupled. Such coupling, in some embodiments, may take place in vivo, by the addition of a single cysteine residue at or around the C-terminus of the ELP. Such ELP components may each be fused to one or more therapeutic components, so as to increase activity or avidity at the target.

### Polynucleotides, Vectors, Host Cells, and Methods for Production

In another aspect, the invention provides polynucleotides comprising a nucleotide sequence encoding the activatable therapeutic agent of the invention. Such polynucleotides may encode an activatable GLP1 or VIP, for example, having Z-N, M-X-N, or M-X¹-X²-N constructs as described. Such polynucleotides may further comprise, in addition to sequences encoding the ELP and therapeutic components, one or more expression control elements. For example, the polynucleotide may comprise one or more promoters or transcriptional enhancers, ribosomal binding sites, transcription termination signals, and polyadenylation signals, as expression control elements. The polynucleotide may be inserted within any suitable vector, which may be contained within any suitable host cell for expression.

In certain embodiments, the host cell is E. coli, and the E. coli is used to produce an activatable protein of the invention having the N-terminal structure X¹-X²-N (as previously described), by expression of a construct encoding M-X¹-X²-N, where Met is removed during expression by the host cell.

Generally, a vector comprising the polynucleotide can be introduced into a cell for expression of the therapeutic agent. The vector can remain episomal or become chromosomally integrated, as long as the insert encoding the therapeutic agent can be transcribed. Vectors can be constructed by standard recombinant DNA technology. Vectors can be plasmids, phages, cosmids, phagemids, viruses, or any other types known in the art, which are used for replication and expression in prokaryotic or eukaryotic cells. It will be appreciated by one of skill in the art that a wide variety of components known in the art (such as expression control elements) may be included in such vectors, including a wide variety of transcription signals, such as promoters and other sequences that regulate the binding of RNA polymerase onto the promoter. Any promoter known to be effective in the cells in which the vector will be expressed can be used to initiate expression of the therapeutic agent. Suitable promoters may be inducible or constitutive.

The invention thereby provides methods of manufacture of recombinant protein therapeutics, including recombinant therapeutics that mimic endogenous proteolytically processed factors (e.g., GLP1). Such products are produced as recombinant proteins by expression of the polynucleotide (e.g., as inserted or introduced into a suitable vector) in a suitable host cell, such as E. coli. The constructs direct expression of biologically active proteins having dipeptidase-sensitive substrates at the N-terminus, as described in connection with Z-N, M-X-N, or M-X¹-X²-N structures. The activatable protein may then be recovered from host cells, and are activatable in vivo or in vitro by peptidase treatment (e.g., DPP-IV treatment).

In certain embodiments, the prodrugs are expressed from E. coli or other bacterial expression system. E. coli may remove N-terminal methionine residues during expression, such that protease sensitive sites are exposed at the N-terminus for administration to a patient. Other expression systems may be employed in accordance with the invention, including yeast expression systems, mammalian cell expression systems, and baculovirus systems. Such expression systems may be used to produce proteins having the DPP substrate M-X-N at the N-terminus as described.

The activatable protein, when employing ELP fusion sequences, may be recovered by inverse temperature cycling. Specifically, as previously described, the ELP component undergoes a reversible inverse phase transition. That is, the ELP components are structurally disordered and highly soluble in water below a transition temperature (Tt), but exhibit a sharp (2-3° C. range) disorder-to-order phase transition when the temperature is raised above the Tt, leading to desolvation and aggregation of the ELP components. For example, the ELP forms insoluble polymers, when reaching sufficient size, which can be readily removed and isolated from solution by centrifugation. Such phase transition is reversible, and isolated insoluble ELPs can be completely resolubilized in buffer solution when the temperature is returned below the Tt of the ELPs. Thus, the therapeutic agents of the invention can, in some embodiments, be separated from other contaminating proteins to high purity using inverse transition cycling procedures, e.g., utilizing the temperature-dependent solubility of the therapeutic agent, or salt addition to the medium. Successive inverse phase transition cycles can be used to obtain a high degree of purity. In addition to temperature and ionic strength, other environmental variables useful for modulating the inverse transition of the therapeutic agents include pH, the addition of inorganic and organic solutes and solvents, side-chain ionization or chemical modification, and pressure.

In certain embodiments, protease (e.g., DPP-IV or other dipeptidyl protease) is used in vitro to manufacture the active molecule.

### Pharmaceutical Compositions

The present invention further provides pharmaceutical compositions comprising the activatable therapeutic agents of the invention (as described above) together with a pharmaceutically acceptable carrier, diluent, or excipient. Such pharmaceutical compositions may be employed in the methods of treatment as described herein.

The therapeutic agents of the invention may overcome certain deficiencies of peptide agents when administered (e.g., parenterally), including in some embodiments, the limitation that such peptides may be easily metabolized by plasma proteases or cleared from circulation by kidney filtration. Traditionally, the oral route of administration of peptide agents may also be problematic, because in addition to proteolysis in the stomach, the high acidity of the stomach destroys such peptide agents before they reach their intended target tissue. Peptides and peptide fragments produced by the action of gastric and pancreatic enzymes are cleaved by exo and endopeptidases in the intestinal brush border membrane to yield di- and tripeptides, and even if proteolysis by pancreatic enzymes is avoided, polypeptides are subject to degradation by brush border peptidases. Any of the peptide agents that survive passage through the stomach are further subjected to metabolism in the intestinal mucosa where a penetration barrier prevents entry into the cells. In certain embodiments, the therapeutic agents of the invention may overcome such deficiencies, and provide compositional forms having enhanced efficacy, bioavailability, therapeutic half-life, persistence, degradation assistance, etc. The therapeutic agents of the invention thus include oral and parenteral dose forms, as well as various other dose forms, by which peptide agents can be utilized in a highly effective manner. For example, in some embodiments, such agents may achieve high mucosal absorption, and the concomitant ability to use lower doses to elicit an optimum therapeutic effect.

The activatable therapeutic agents of the present invention, where ELP sequences are employed, may be administered in smaller doses and/or less frequently than native sequences. While one of skill in the art can determine the desirable dose in each case, a suitable dose of the therapeutic agent for achievement of therapeutic benefit, may, for example, be in a range of about 1 microgram (μg) to about 100 milligrams (mg) per kilogram body weight of the recipient per day, preferably in a range of about 10 μg to about 50 mg per kilogram body weight per day and most preferably in a range of about 10 μg to about 50 mg per kilogram body weight per day. The desired dose may be presented as one dose or two or more sub-doses administered at appropriate intervals throughout the day. These sub-doses can be administered in unit dosage forms, for example, containing from about 10 μg to about 1000 mg, preferably from about 50 μg to about 500 mg, and most preferably from about 50 μg to about 250 mg of active ingredient per unit dosage form. Alternatively, if the condition of the recipient so requires, the doses may be administered as a continuous infusion.

The mode of administration and dosage forms will of course affect the therapeutic amount of the peptide active therapeutic agent that is desirable and efficacious for a given treatment application. For example, orally administered dosages can be at least twice, e.g., 2-10 times, the dosage levels used in parenteral administration methods.

The therapeutic agents of the invention may be administered per se as well as in various forms including pharmaceutically acceptable esters, salts, and other physiologically functional derivatives thereof. The present invention also contemplates pharmaceutical formulations, both for veterinary and for human medical use, which include therapeutic agents of the invention. In such pharmaceutical and medicament formulations, the therapeutic agents can be used together with one or more pharmaceutically acceptable carrier(s) therefore and optionally any other therapeutic ingredients. The carrier(s) must be pharmaceutically acceptable in the sense of being compatible with the other ingredients of the formulation and not unduly deleterious to the recipient thereof. The therapeutic agents are provided in an amount effective to achieve the desired pharmacological effect, as described above, and in a quantity appropriate to achieve the desired daily dose.

The formulations of the therapeutic agent include those suitable for parenteral as well as non-parenteral administration, and specific administration modalities include oral, rectal, buccal, topical, nasal, ophthalmic, subcutaneous, intramuscular, intravenous, transdermal, intrathecal, intra-articular, intra-arterial, sub-arachnoid, bronchial, lymphatic, vaginal, and intra-uterine administration. Formulations suitable for oral and parenteral administration are preferred.

When the therapeutic agent is used in a formulation including a liquid solution, the formulation advantageously can be administered orally or parenterally. When the therapeutic agent is employed in a liquid suspension formulation or as a powder in a biocompatible carrier formulation, the formulation may be advantageously administered orally, rectally, or bronchially.

When the therapeutic agent is used directly in the form of a powdered solid, the active agent can be advantageously administered orally. Alternatively, it may be administered bronchially, via nebulization of the powder in a carrier gas, to form a gaseous dispersion of the powder which is inspired by the patient from a breathing circuit comprising a suitable nebulizer device.

The formulations comprising the therapeutic agent of the present invention may conveniently be presented in unit dosage forms and may be prepared by any of the methods well known in the art of pharmacy. Such methods generally include the step of bringing the therapeutic agents into association with a carrier which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation.

Formulations suitable for oral administration may be presented as discrete units such as capsules, cachets, tablets, or lozenges, each containing a predetermined amount of the active ingredient as a powder or granules; or a suspension in an aqueous liquor or a non-aqueous liquid, such as a syrup, an elixir, an emulsion, or a draught.

A tablet may be made by compression or molding, optionally with one or more accessory ingredients. Compressed tablets may be prepared by compressing in a suitable machine, with the therapeutic agent being in a free-flowing form such as a powder or granules which optionally is mixed with a binder, disintegrant, lubricant, inert diluent, surface active agent, or discharging agent. Molded tablets comprised of a mixture of the powdered peptide active therapeutic agent-ELP construct(s) with a suitable carrier may be made by molding in a suitable machine.

A syrup may be made by adding the peptide active therapeutic agent-ELP construct(s) to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration conveniently comprise a sterile aqueous preparation of the therapeutic agent, which preferably is isotonic with the blood of the recipient (e.g., physiological saline solution). Such formulations may include suspending agents and thickening agents or other microparticulate systems which are designed to target the peptide active therapeutic agent to blood components or one or more organs. The formulations may be presented in unit-dose or multi-dose form.

Nasal spray formulations comprise purified aqueous solutions of the therapeutic agent with preservative agents and isotonic agents. Such formulations are preferably adjusted to a pH and isotonic state compatible with the nasal mucus membranes.

Formulations for rectal administration may be presented as a suppository with a suitable carrier such as cocoa butter, hydrogenated fats, or hydrogenated fatty carboxylic acid.

Topical formulations comprise the therapeutic agent dissolved or suspended in one or more media, such as mineral oil, petroleum, polyhydroxy alcohols, or other bases used for topical pharmaceutical formulations.

In addition to the aforementioned ingredients, the formulations of this invention may further include one or more accessory ingredient(s) selected from diluents, buffers, flavoring agents, disintegrants, surface active agents, thickeners, lubricants, preservatives (including antioxidants), and the like.

The features and advantages of the present invention are more fully shown with respect to the following non-limiting examples.

### EXAMPLES

### Example 1

### Activatable GLP1

Constructs of GLP1-ELP with DPP-IV sensitive sites were made and the proteins from these constructs were expressed in E. coli. FIG. 1 illustrates an exemplary activatable GLP1 protein of the invention. FIG. 1A is a GLP1 containing Ala-Ala at the N-terminus, which is removed in vivo by peptidase processing to expose the natural N-terminal His of GLP1(7-37). The molecule further contains a substitution of Gly at position 8 (position 2 with respect to N-terminal His), to prevent unwanted proteolysis. The exemplary molecule further comprises an ELP fusion at the C-terminus to extend half-life. The ELP fusion sequence, designated as ELP1-120, comprises 12 repeats of an ELP1 motif (VPGXG) where X=V₅G₃A₂. FIG. 1B illustrates the same molecule after peptidase processing.

DPP-IV cleaves the N-terminus dipeptides containing proline, alanine or serine in the second position. Although, it is theoretically possible to make constructs containing Met-Pro, Met-Ala or Met-Ser before the histidine as DPP-IV sensitive sites, E. coli tends to remove the N-terminal methionine from proteins containing alanine and serine (and sometimes proline) leaving only one amino acid before the histidine. Remaining one amino acids are no longer substrates for DPP-IV. Exemplary constructs include the following N-terminal sequences before the natural N-terminal His of GLP-1:

Met-Pro (requires Met for DPP-IV digestion)

Met-Ala-Ala (Met is removed by E. coli)

Met-Ala-Pro (Met is removed by E. coli)

Met-Ser-Pro (Met is removed by E. coli)

Met-Ser-Ala (Met is removed by E. coli).

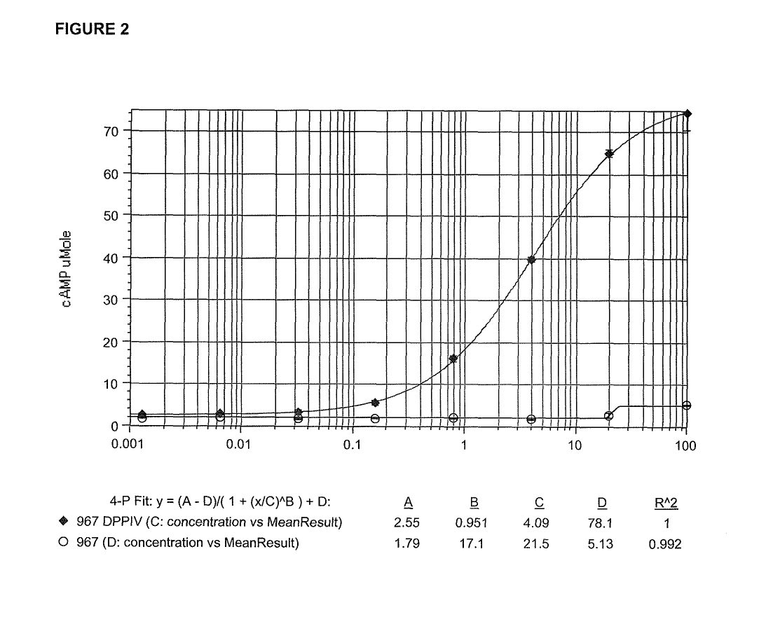

The protein from the construct containing Met-Ala-Ala at the N-terminus was expressed and purified. The purified protein was tested in an in vitro biological assay to measure the activity of GLP1-ELP before and after treatment with DPP-IV (FIG. 2).

FIG. 2 shows cAMP production by CHO cells containing human GLP1 receptor. These cells respond to the increasing concentrations of GLP1 and its active analogues x-axis) by producing cAMP. PB0967 (designated 967 on the graph (FIG. 1)) is a GLP1-ELP construct with Met-Ala-Ala at the N-terminus. It is anticipated that the Met is removed by E. coli during expression. As shown in this graph the protein is initially inactive and is activated if it is first treated with DPP-IV to remove the remaining Ala-Ala and expose the N-terminal His of GLP1.

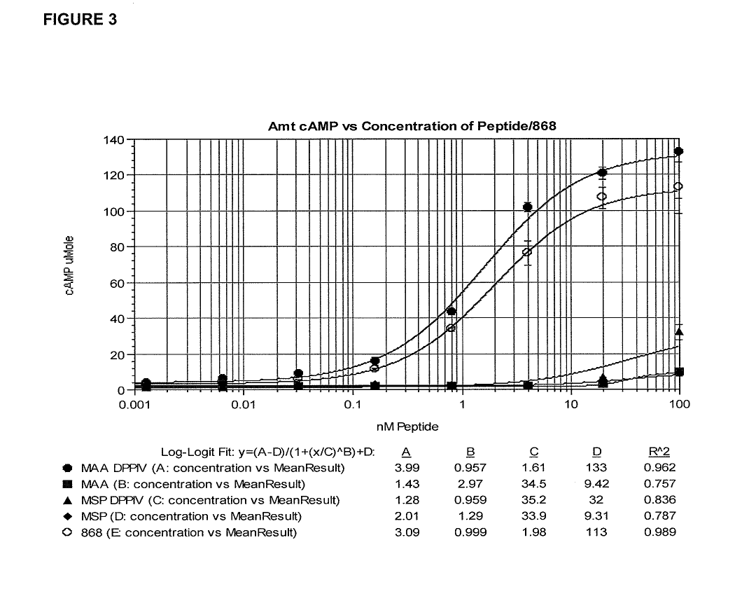

FIG. 3 shows the results of a cAMP assay comparing two GLP1 constructs with MAA and MSP at the N-terminus before His⁷, respectively. FIG. 3 shows the results with protein treated with rDPP-IV, untreated protein, and with PB0868 (GLP1-ELP1-90). For comparison, in this assay Exendin-4 peptide has an EC50 of around 1 nM.

### Example 2

### In Vivo Activation of GLP1

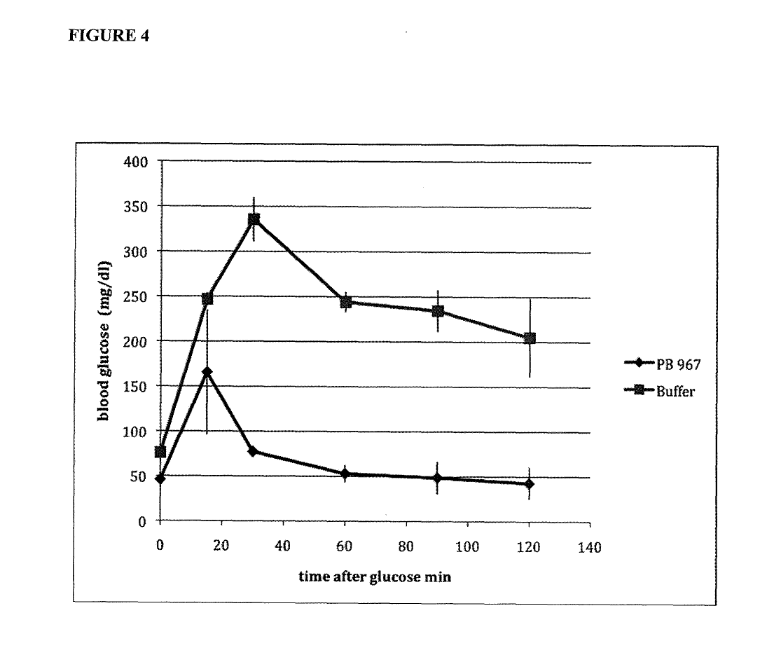

FIG. 4 shows IPGTT in normal mice 12 hours after injection of PB967 (dose was about 30 nmol/Kg) or buffer. The results demonstrate that injection of PB967 provides reduction in glucose excursion and rapid recovery to baseline. PB967 was therefore processed in vivo to the active form.

### Example 3

### PB1047 Activity

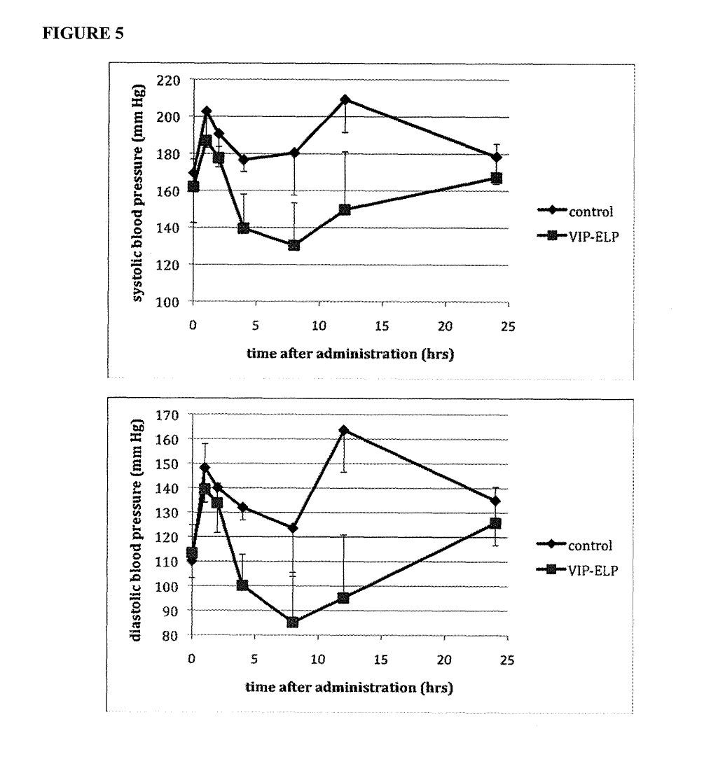

This example measures blood pressure changes in response to PB1047 (maa VIP ELP1-120). In this example, Spontaneously Hypertensive (SH) rats were injected SQ with 10 mg/kg of VIP-ELP (PB1047) or buffer (control) and their blood pressure was monitored over 24 hour period. The animals used for this study were approximately 12 weeks of age and had systolic blood pressures averaging 160-170 mmHg. The upper panel of FIG. 5 shows the changes in systolic blood pressure and the bottom panel shows diastolic pressure. Each time point represents the average blood pressure of 5 animals with Standard Deviation.

As this example shows, PB1047 treated animals showed a significant difference at 4 hours post injection both in their systolic and diastolic pressure compared to controls. The difference in blood pressure between controls and treated persisted until 12 hours after injection.

The contents of all references, patents and published patent applications cited throughout this application, as well as the Figures and the Sequence Listing, are incorporated herein by reference for all purposes.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

Although the application has been broken into sections to direct the reader's attention to specific embodiments, such sections should be not be construed as a division amongst embodiments. The teachings of each section and the embodiments described therein are applicable to other sections.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A protein comprising a therapeutic protein and a substrate sensitive to dipeptidyl peptidase (DPP) at the N-terminus of the protein.

2. The protein of claim 1, wherein the dipeptidyl peptidase (DPP) activates or increases the activity of the therapeutic protein by cleavage of the substrate.

3. The protein of claim 1, wherein the therapeutic protein is a recombinant version of a protein factor that is processed from a native precursor molecule in vivo.

4. The protein of claim 1, wherein the therapeutic protein is a hormone, chemokine, neuropeptide, or vasoactive peptide.

5. The protein of claim 1, wherein the therapeutic protein is GLP-receptor agonist.

6. The protein of claim 5, wherein the GLP-receptor agonist is a GLP1.

7. The protein of claim 6, wherein the GLP1 is GLP1(7-37 A8G)

8. The protein of claim 1, wherein the therapeutic protein is vasoactive intestinal peptide.

9. The protein of claim 1, wherein the therapeutic protein requires an N-terminal amino acid other than methionine for activity.

10. The protein of claim 1, wherein the N-terminal amino acid of the therapeutic protein is His or other amino acid that limits the removal of an N-terminal methionine by E. coli.

11. The protein of claim 1, wherein the substrate is sensitive to one or more of DPP-I, DPP-III, DPP-IV, DPP-VI, DPP-VII, DPP-VIII, DPP-IX, and DPP-X.

12. The protein of claim 11, wherein the substrate is sensitive to DPP-IV.

13. The protein of claim 1, wherein the protein has an N-terminal sequence of the formula X1-X2-N, where: X1 is selected from Gly, Ala, Ser, Cys, Thr, Val, and Pro; and X2 is selected from Pro, Ala, and Ser, and N is the desired N-terminus of the biologically active molecule.

14. The protein of claim 13, wherein X1 is Pro, Ala or Ser, and X2 is Ala or Pro.

15. The protein of claim 13, wherein N is His.

16. The protein of claim 1, wherein the protein has an N-terminal sequence of the formula M-X-N, where: M is methionine; X is Pro, Ala, or Ser; and N is the N-terminus of the biologically active molecule.

17. The protein of claim 16, where N is His.

18. The protein of claim 1, further comprising a C-terminal ELP fusion.

19. (canceled)

20. A method of treating a condition, disorder, or disease in a mammalian patient, comprising, administering the protein of claim 1 to a patient in need.

21. (canceled)

22. (canceled)

23. A method of producing the protein of claim 1, comprising, expressing the protein in a host cell, and recovering the protein.

24. The method of claim 23, wherein the host cell is E. coli.

25. (canceled)

26. (canceled)

27. (canceled)

28. (canceled)
